(19) United States
(12) Patent Application Publication
(10) Pub. No.: US 2011/0245667 A1
(43) Pub. Date: Oct. 6, 2011

(54) COMPRESSION DEVICE USED IN ULTRASONIC MEASUREMENT, PRESSING CONTROL METHOD THEREOF, AND PHOTOACOUSTIC MEASUREMENT APPARATUS

(75) Inventor: Toshinobu Tokita, Yokohama-shi (JP)

(73) Assignee: CANON KABUSHIKI KAISHA, Tokyo (JP)

(21) Appl. No.: 13/131,975

(22) PCT Filed: Jan. 20, 2010
(86) PCT No.: PCT/JP2010/050994

§ 371 Date: Jun. 20, 2011

(30) Foreign Application Priority Data
Jan. 21, 2009 (JP) 2009-010629

(51) Int. Cl. A61B 8/00; A61B 6/00

(52) U.S. Cl. 600/437; 600/407

(57) ABSTRACT

A compression device for compressing a subject used in ultrasonic measurement that receives ultrasonic waves from a subject to acquire biological information of the subject, comprises: two compression plates used when compressing the subject, and face each other in a vertical direction with respect to a horizontal plane; a flexible member arranged so as to cover and seal an area between both side surfaces of and a bottom surface between the two compression plates, and so as not cover an area between top surfaces of the two compression plates such that an upper portion is open; a compression mechanism that relatively changes a distance between the two compression plates to sandwich and compress the subject; and a piping unit that supplies a matching liquid to a space portion surrounded by the two compression plates and the flexible member, and discharges the matching liquid that has been supplied.

### TECHNICAL FIELD

The present invention relates to a compression device used in ultrasonic measurement and a pressing control method thereof, and a photoacoustic measurement apparatus.

More particularly, the present invention relates to a compression device used in a photoacoustic measurement apparatus that radiates near infrared rays to inside a subject, receives photoacoustic waves generated inside the subject with an ultrasound probe, and displays a tissue image of inside the subject.

### BACKGROUND ART

Non-Patent Document 1 discusses a photoacoustic measurement apparatus that has been developed for use in breast cancer examination.

According to Non-Patent Document 1, a subject (breast) is compressed between a glass plate and an ultrasound probe, and illumination light (near infrared rays) that employs an Nd:YAG laser as a light source is radiated at the breast through the glass plate.

Photoacoustic waves generated inside the subject (breast) are received with the ultrasound probe. The received photoacoustic waves are used to reconstruct an image of tissue inside the subject (breast), particularly angiogenesis in breast cancer, and the reconstructed image is displayed.

However, according to the apparatus discussed in Non-Patent Document 1, gaps are caused due to dead spaces between a part of the subject (breast) and the ultrasound probe in which the subject (breast) cannot contact the ultrasound probe. The gap portions consist of air. Since the acoustic impedance of air is significantly different from the acoustic impedance inside a subject, photoacoustic waves are not transmitted through the gap portions and regions arise with respect to which image reconstruction cannot be performed.

Therefore, according to Patent Document 1, a transmission-type ultrasonic measurement apparatus is proposed that suppresses the occurrence of regions for which image reconstruction cannot be performed by filling gap portions with an acoustic matching medium formed of water or a gel, to thereby match the acoustic impedances.

The ultrasonic measurement apparatus according to Patent Document 1 will now be described using FIGS. 9A and 9B.

In FIGS. 9A and 9B, a compression device 20 compresses a breast as a subject 81. A bellows mechanism 60 is attached to the compression device 20.

An acoustic matching medium 80 for matching acoustic impedances is filled inside the compression device 20.

The ultrasonic measurement apparatus also includes a transmitting transducer 121 and a receiving transducer 122.

The acoustic matching medium 80 is sealed by the compression device 20 and the bellows mechanism 60, and the compression device 20 is movable in a compressing direction.

[Non-Patent Document 1] Srirang Manohar, et al., The Twente photoacoustic mammoscope: system overview and performance, Physics in Medicine and Biology 50 (2005) 2543-2557.

[Patent Document 1] Japanese Patent Application Laid-Open No. S60-190853

However, there are the following problems with the ultrasonic measurement apparatus of Patent Document 1 that is the conventional example as described above.

Although according to Patent Document 1 a problem does not arise in which the acoustic impedance differs because of gap portions and regions arise that cannot be measured as in the case of Non-Patent Document 1 described above, a problem remains relating to retention of a matching liquid (acoustic matching medium 80).

That is, as illustrated in FIGS. 9A and 9B, when a form is adopted in which a subject is inserted from the side, when water is employed as a matching liquid (acoustic matching medium 80) there is a risk that the matching liquid (acoustic matching medium 80) will leak before and after compressing by the compression device 20.

Alternatively, when a gel is employed as the matching liquid (acoustic matching medium 80), there is a risk that the matching liquid (acoustic matching medium 80) will overflow.

There is no reference to these solutions in Patent Document 1.

### DISCLOSURE OF THE INVENTION

The present invention was made in view of the above problems, and an object of the invention is to provide, as a compression device for compressing a subject that is used in ultrasonic measurement that receives ultrasonic waves from the subject to acquire biological information of the subject, a compression device that can match an acoustic impedance with a subject and can suppress leaking or overflowing of a matching liquid, as well as a compressing control method thereof, and a photoacoustic measurement apparatus.

The present invention is directed to a compression device for compressing a subject that is used in ultrasonic measurement that receives ultrasonic waves from a subject to acquire biological information of the subject, comprising:

two compression plates that are used when compressing the subject, and that face each other in a vertical direction with respect to a horizontal plane;

a flexible member arranged so as to cover and seal an area between both side surfaces of and a bottom surface between the two compression plates, and so as not cover an area between top surfaces of the two compression plates such that an upper portion is open;

a compression mechanism that relatively changes a distance between the two compression plates to sandwich and compress the subject; and a piping unit that supplies a matching liquid to a space portion surrounded by the two compression plates and the flexible member, and discharges the matching liquid that has been supplied.

The compression device can comprise a detection portion that detects a supply state of the matching liquid that is supplied to the space portion.

The compression device can comprise a control portion that controls supply of the matching liquid so that the matching liquid does not overflow from the space portion, based on an output of the detection portion.

The flexible member can be any member of the group comprising an accordion-like member, a bellows, a resin film, or a rubber sheet.

The piping unit can comprise at least one member of the group comprising:

a valve for controlling supply or discharge of the matching liquid;

a tank for storing the matching liquid;

a pump for feeding the matching liquid;

a pressure and flow rate adjustment valve for adjusting a pressure or a flow rate of the matching liquid;

a heater for heating the matching liquid;

a filter for removing impurities from the matching liquid; and

a deaerated water generation apparatus that deaerates dissolved gas included in the matching liquid.

The piping unit can comprise a valve for controlling supply of a washing liquid.

The present invention is directed to a photoacoustic measurement apparatus that comprises a compression device which sandwiches and compresses a subject between two compression plates, and that compresses a subject using the compression device and detects as a photoacoustic signal an ultrasonic wave generated by a light irradiated at the subject, characterized in that the compression device is a compressing unit comprising the compression device; the photoacoustic measurement apparatus further comprising:

an illumination light optical system for irradiating the light at a subject through the compression plate;

a probe that receives a photoacoustic signal generated from the subject through the compression plate; and

a processing portion that processes a photoacoustic signal received by the probe to reconstruct an image.

In the photoacoustic measurement apparatus, of the two compression plates, a compression plate on a side on which the probe is disposed can be formed by resin.

The resin can comprise polymethylpentene.

The present invention is directed to a compressing control method of a compression device for compressing a subject that is used in ultrasonic measurement that receives ultrasonic waves from the subject to acquire biological information of the subject, comprising:

a washing process of washing inside of the space portion of the compression device according to any one of claims 1 to 6; and

after the washing process, a compressing process of sandwiching and compressing the subject with the two compression plates.

The washing process can comprise: discharging a matching liquid that has been supplied to the space portion;

supplying a washing liquid to inside the space portion;

after supplying the washing liquid, discharging the washing liquid that has been supplied to inside the space portion; and

after discharging the washing liquid, supplying a matching liquid to inside the space portion.

The compressing process can comprise:

detecting setting of the subject inside the space portion;

starting a compressing operation by a compression mechanism in order to compress the subject;

controlling a liquid volume of a matching liquid that is filled to inside the space portion; and

ending the compressing operation by the compression mechanism.

Based on an output of a detection portion that detects a supply state of the matching liquid that is supplied to the space portion, the control of a liquid volume of the matching liquid can control a supply of the matching liquid so that the matching liquid does not overflow from the space portion.

According to the present invention it is possible to realize, as a compression device for compressing a subject that is used in ultrasonic measurement that receives ultrasonic waves from the subject to acquire biological information of the subject, a compression device that can match an acoustic impedance with a subject and can suppress leaking or overflowing of a matching liquid, as well as a compressing control method thereof, and a photoacoustic measurement apparatus.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a view that describes a configuration of a compression device when released according to Embodiment 1 of the present invention;

FIG. 2 is a view that illustrates the configuration of the compression device when performing a compressing operation according to Embodiment 1 of the present invention;

FIG. 3 is a view that describes a recirculation-type piping unit according to Embodiment 1 of the present invention;

FIG. 4 is a view that describes the discharge and supply of a matching liquid from and to the inside of the compression device according to Embodiment 1 of the present invention, as well as washing of the inside of the compression device;

FIG. 5 is a flowchart that describes a washing process that washes the inside of a compression device according to Embodiment 2 of the present invention;

FIG. 6 is a flowchart that describes a compressing process performed by the compression device according to Embodiment 2 of the present invention;

FIG. 7 is a flowchart that describes a compressing process performed by the compression device according to Embodiment 2 of the present invention, that is different to the method of the flowchart illustrated in FIG. 6;

FIG. 8 is a view that describes a photoacoustic measurement apparatus on which a compression device is mounted according to Embodiment 3 of the present invention; and

FIGS. 9A and 9B are views that describe the background art according to a conventional example.

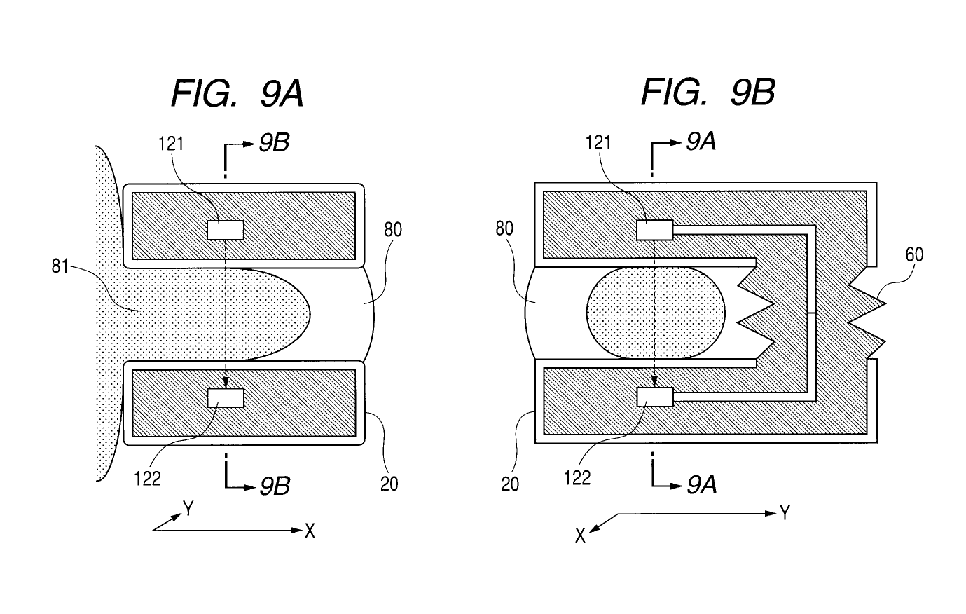

The reference numerals shown in the drawings are defined as follows.
1: compression device
2: compression plate
3: compression plate fastening portion
4: guide
5: compression mechanism
6: bellow
7: piping unit
8: matching liquid
9: liquid volume sensor
10: illumination light optical system
11: illumination light scan unit
12: probe
13: probe scan unit

### DESCRIPTION OF THE EMBODIMENTS

Preferred embodiments of the present invention will now be described in detail in accordance with the accompanying drawings.

Embodiments of the present invention are described hereunder.

A compression device of the present embodiments is a compression device for compressing a subject that is used in ultrasonic measurement that receives ultrasonic waves from a subject to acquire biological information of the subject. The compression device is configured as follows.

More specifically, in order to enable matching of an acoustic impedance with that of a subject and suppress leaking or overflowing of a matching liquid, as well as to enable insertion and compressing of a subject, a compression device that includes a space portion for filling a matching liquid is configured such that an upper part thereof is open.

To achieve this configuration, the compression device includes two compression plates that face each other in a vertical direction with respect to a horizontal plane, and that are used when compressing the subject; and a flexible member arranged so as to cover and seal an area between both side surfaces of and a bottom surface between the two compression plates that face each other in the vertical direction, and also so as not to cover an area between upper surfaces of the two compression plates so that an upper part of the compression device is open.

The compression device also includes a compression mechanism that relatively changes a distance between the two compression plates facing each other in the vertical direction so as to sandwich and compress the subject; and a piping unit that is disposed in the space portion surrounded by the two compression plates and the flexible member arranged between both side surfaces of and a bottom surface between the two compression plates, and that supplies a matching liquid and discharges the matching liquid that has been supplied.

According to this configuration, it is possible to reduce leaking or overflowing of a matching liquid for matching acoustic impedances from the compression device, and compress a subject. Furthermore, by mounting the compression device to a photoacoustic apparatus, since leaking or overflowing of a matching liquid is reduced, time and labor required for cleaning can be decreased and the operational availability rate of the photoacoustic apparatus can be improved. Further, dead space that cannot be measured can be simultaneously decreased.

The compression device of the present invention is used in ultrasonic measurement that receives ultrasonic waves from a subject to acquire biological information of the subject, and for example is mounted to the following type of apparatus. The apparatus is a photoacoustic measurement apparatus that radiates pulsed light at a subject and receives ultrasonic waves emitted due to a local temperature increase that occurs when the pulsed light is absorbed. This is referred to as so-called PAT (Photo Acoustic Tomography), and ultrasonic waves generated from a subject are also referred to as photoacoustic waves.

Since light is attenuated significantly in vivo, a compression device such as that of the present invention can be suitably applied for performing photoacoustic measurement as far as a deep portion of a subject. The compression device of the present invention can also be applied to a known ultrasound apparatus that radiates ultrasonic waves at a subject and receives ultrasonic waves (reflection echo) reflected from the subject.

In the present specification, the term “matching liquid” refers to a material that is supplied between a probe that receives ultrasonic waves and a subject, and has an acoustic impedance value that is approximate to that of the subject. As long as the material has no fixed shape and cannot be retained without a structure such as that of the present invention, the concept also includes a gel and the like.

### EMBODIMENTS

Hereunder, embodiments of the present invention are described.

### Embodiment 1

According to Embodiment 1, a configuration example of a compression device of the present invention is described.

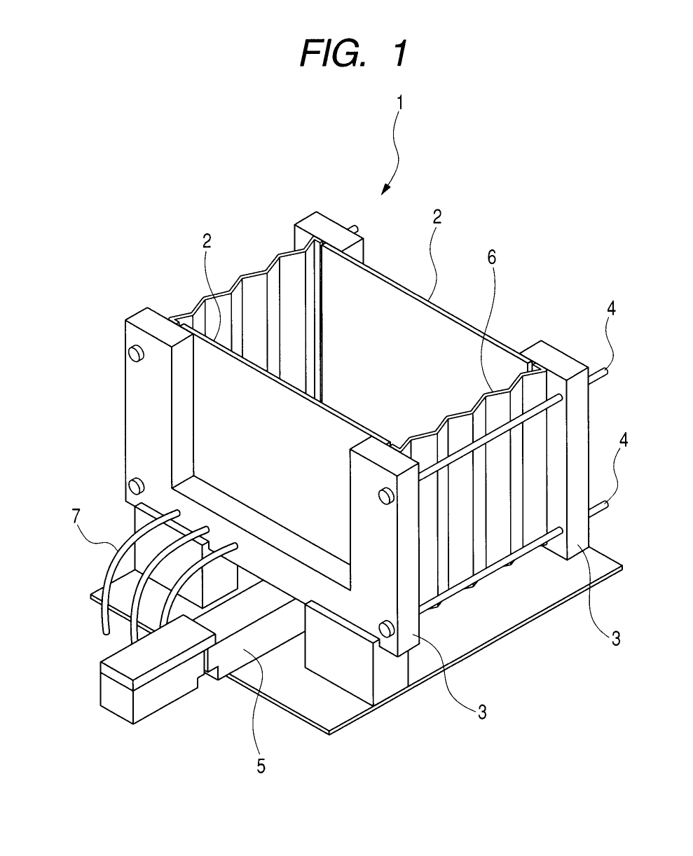

FIG. 1 illustrates a view that describes the configuration of a compression device when released according to the present embodiment. The configuration illustrated in FIG. 1 includes a compression device 1, compression plates 2, compression plate fastening portions 3, guides 4, a compression mechanism 5, bellow 6 and a piping unit 7.

In the compression device 1 of this embodiment, the compression plates 2 for sandwiching a subject are composed of two flat plates that face each other in a vertical direction with respect to a horizontal plane. The two flat plates can be composed by parallel flat plates.

The compression plate fastening portions 3 are members for retaining the compression plates 2. A plurality of guides 4 and a compression mechanism 5 that employs a robot mechanism are connected to the compression plate fastening portions 3.

These components are used to insert a subject inside the compression device 1 and compress the subject.

Although according to the present embodiment the compression mechanism 5 is constituted by a robot mechanism, the compression mechanism 5 is not limited thereto, and may be an air cylinder mechanism or a manual mechanism (vise mechanism) that uses a rack-and-pinion or a worm gear. Further, although an example in which four guides 4 are provided is illustrated in the drawings, the number of the guides 4 is not limited thereto.

The compression device 1 of the present embodiment includes a space portion in which an area between two side surfaces of the aforementioned two flat plates facing each other in the vertical direction and a bottom surface surrounded by these two side surfaces is sealed by a flexible member, and in which an upper part is open.

The flexible member in this case is constituted by a bellows 6 according to the present embodiment. End portions of the bellows 6 are fixed to the compression plate fastening portions 3 so as to surround each of the side surfaces of the compression plates 2 and the bottom surface.

Although, as illustrated in FIG. 1, the bellows 6 is formed in an angular shape according to the present embodiment, the shape of the bellows 6 is not limited thereto, and the bellows 6 may be a round shape.

As the material of the bellows 6, rubber, molded resin, or a metal such as stainless steel can be used. For example, the bellows may also be obtained by cutting off a part of a welded bellows.

Further, a configuration may be adopted in which the bellows 6 is not used, and instead a flexible member such as a resin film or a rubber sheet is used, in which the ends thereof are fixed to the compression plate fastening portions 3.

Further, in consideration of mechanical strength, a configuration is adopted in which the compression plates 2 are supported by the compression plate fastening portions 3, and end portions of the bellows 6 are fixed to the side surfaces and bottom surfaces of the compression plate fastening portions 3. However, the configuration is not limited thereto. A configuration may also be adopted in which ends of the bellows 6 are fixed directly to the compression plates 2 so as to cover the side surfaces of and the bottom surface between the two compression plates 2 that face each other.

In that case, the configuration is effective even if the compression plate fastening portions 3 are eliminated therefrom, and the guides 4 or the compression mechanism 5 may also be directly attached to the compression plates 2.

A portion of a piping unit 7 is illustrated in FIG. 1. Piping that supplies and recovers a matching liquid is provided inside the above described space portion in the compression device 1. Further, piping that supplies a washing liquid for washing inner walls of the above described space portion in the compression device 1 can be provided in the piping unit 7.

According to the above described configuration, because the above described space portion in the compression device 1 is sealed by the bellows 6, when a subject is inserted into the space portion from an opening in the upper part thereof and matching liquid is filled therein, the matching liquid does not leak.

As a result, it is possible to perform a compressing operation with the compression plates 2 using the compression mechanism 5.

Further, since the matching liquid enters between the compression plates 2 and an unshown subject, gaps caused by dead spaces do not arise, and the acoustic impedance can be matched with that of the subject.

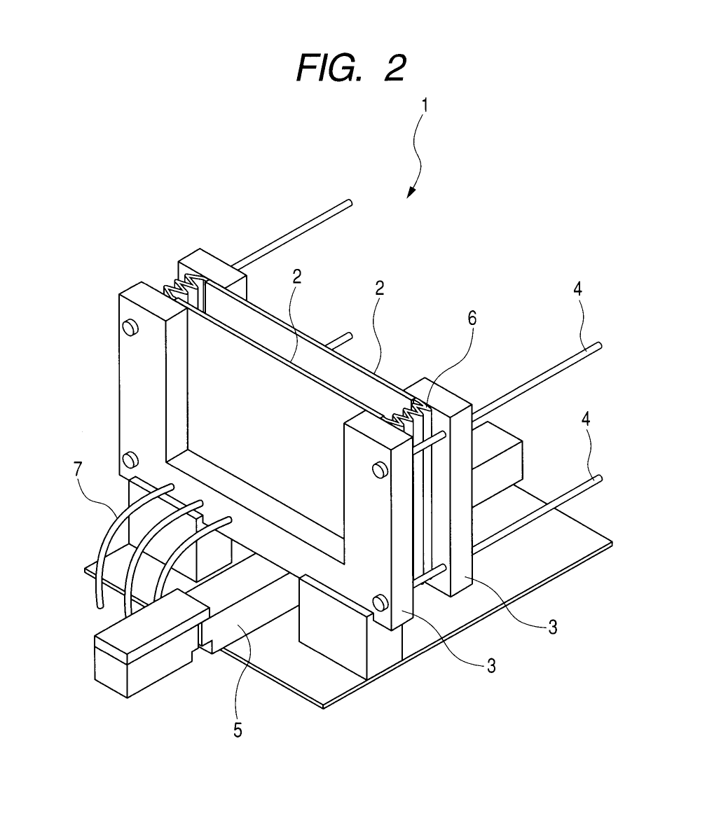

FIG. 2 is a view that illustrates the configuration of the compression device at a time of a compressing operation according to the present embodiment.

At a time of a compressing operation, the compression device 1 of the present embodiment is changed from a state in which the compression plates 2 are open as illustrated in FIG. 1 to a state in which the compression plates 2 are subjected to a compressing operation by the compression mechanism 5 and compress the subject (unshown).

Next, the piping unit 7 is described in detail.

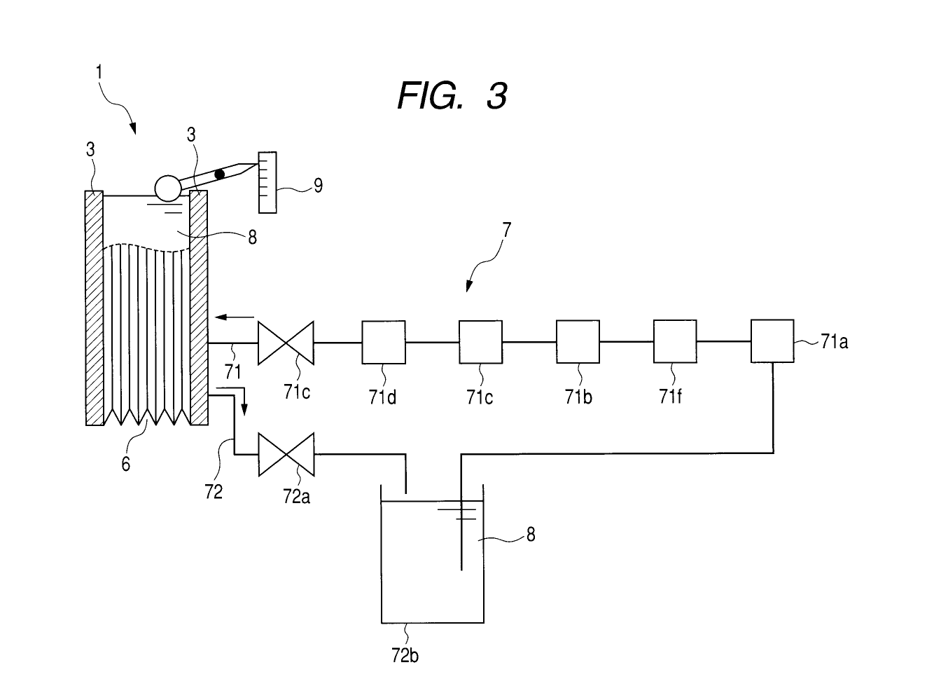

FIG. 3 illustrates a view that describes the piping unit 7 according to the present embodiment.

The configuration illustrated in FIG. 3 includes the piping unit 7, a matching liquid 8, and a liquid volume sensor 9 as a detection portion that detects the supply state of matching liquid that is supplied to the space portion.

In the present embodiment, the matching liquid 8 is filled inside the compression device in order to match the acoustic impedance of photoacoustic waves.

Further, the liquid volume sensor 9 is provided for measuring the liquid volume (liquid surface) of the matching liquid 8 inside the compression device 1.

The piping unit 7 includes at least a supply line 71 that supplies the matching liquid 8 and a recovery line 72 that recovers the matching liquid 8.

A valve 72a is provided in the recovery line 72, and the matching liquid 8 is stored in a tank 72b.

The pressure or flow rate of a matching liquid that is drawn up and fed by the pump 71a from the tank 72b is adjusted by an opening/closing operation of a pressure and flow rate adjustment valve 71b.

The matching liquid 8 whose pressure or flow rate has been adjusted is heated by a heater 71c to a temperature of around 40° C. that is approximate to the temperature of the subject, and is supplied to inside the compression device 1 via a filter 71d for eliminating impurities.

When the matching liquid 8 is water, it is desirable to deaerate dissolved gas. In this case, a deaerated water generation apparatus is incorporated as a deaeration apparatus 71f in one portion of the piping unit 7.

According to the above described configuration, the liquid volume of the matching liquid 8 inside the compression device 1 is controlled by opening/closing of the valve or by adjusting the pressure or flow rate of the matching liquid 8 using the pressure and flow rate adjustment valve 71b. More specifically, these components function as a control portion that controls the supply of the matching liquid.

At this time, the control portion measures the liquid volume (liquid surface) of the matching liquid 8 with the liquid volume sensor 9, and feeds back the output thereof.

In particular, since the volume of the compression device 1 decreases when the compression device 1 is compressed by the compression mechanism 5, the liquid surface inside the compression device 1 rises.

Since the matching liquid 8 will overflow from the compression device 1 when the liquid surface rises, the control portion opens the valve 72a of the matching liquid recovery line 72 based on an output according to a measurement result of the flow rate sensor 9.

In contrast, since the volume of the compression device 1 increases when the compression device 1 is caused to open by the compression mechanism 5, the control portion feeds back the output of the liquid volume sensor 9, adjusts the pressure and flow rate adjustment valve 71b, and increases the supply pressure and supply flow rate of the matching liquid 8. In this manner, the control portion controls the supply of a matching liquid so that the matching liquid does not overflow from the space portion based on the output of the liquid volume sensor 9 as a detection portion.

Although control of the supply and recovery of the matching liquid 8 based on output of the liquid volume sensor 9 has been described in the foregoing, the present invention is not limited thereto.

For example, since the compression device 1 uses the bellows 6, changes in the volume within the compression device 1 accompanying a change in the distance between the compression plates 2 are replicated.

Accordingly, a configuration may be adopted in which supply and recovery of the matching liquid 8 is controlled by providing a compressing distance sensor (unshown) in the compression device 1 and calculating changes in the volume of the compression device 1 based on the output thereof to serve as the liquid volume sensor 9. In this case, the compressing distance sensor corresponds to the detection portion of the present invention.

Further, hygiene is ensured by boiling the matching liquid 8 in the tank 72b to sterilize the matching liquid 8 and eliminate bacteria.

The order of the elements inside the piping unit 7 is not limited to the order illustrated in FIG. 3.

According to the above described configuration, if the matching liquid 8 is caused to circulate it is not necessary to prepare a utility for filling and discharging the matching liquid 8, and thus the conditions for installing an apparatus on which the compression device 1 is mounted are eased.

Although it has been described thus far that the matching liquid 8 is stored once in the tank 72b after recovery, the present invention is not limited thereto.

Since the matching liquid 8 contacts the subject, in some cases it is preferable to discharge the matching liquid 8 without using the matching liquid 8 again when examining the next person.

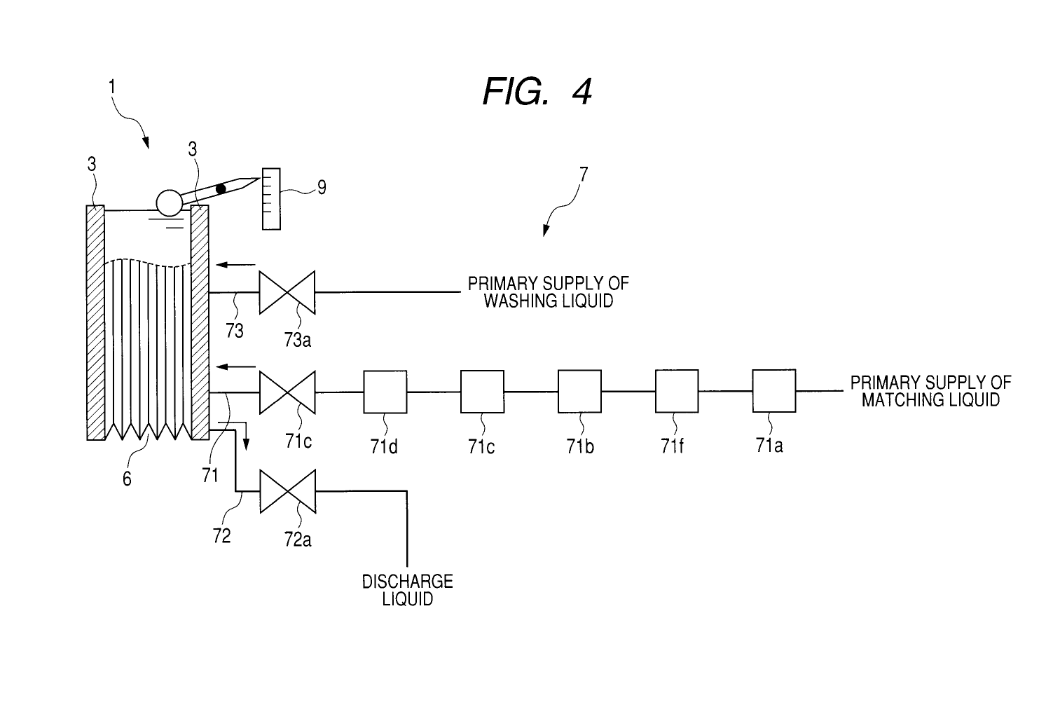

FIG. 4 is a system diagram of a configuration that can discharge matching liquid 8 and supply a new matching liquid 8. Accordingly, the liquid recovery line 72 is connected to liquid to be discharged, and the matching liquid supply line 71 is introduced from a primary supply.

A washing liquid supply line 73 is also provided so that a washing liquid can be supplied to inside the compression device 1. In this connection, the washing liquid supply line 73 may also be provided in the configuration illustrated in FIG. 3. However, it is better to also separately provide a line to recover the washing liquid, so that the matching liquid 8 and the washing liquid do not mix.

According to the above configuration, although a utility is required for filling and discharging the matching liquid 8, the configuration is more hygienic because matching liquid 8 that has been in contact with another subject is not filled into the compression device 1. Further, since the compression device 1 can be washed with washing liquid, the hygiene of the compression device is further improved.

### Embodiment 2

In Embodiment 2, a configuration example of a compressing control method of a compression device used in photoacoustic measurement of the present invention is described.

First, before examining a subject (for example, a breast), washing steps (S51 to S54) for washing the inside of the compression device are performed in order.

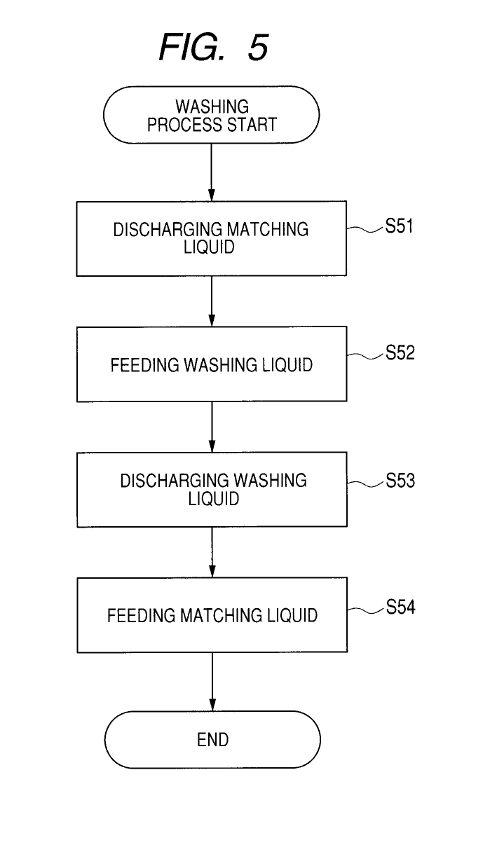

FIG. 5 illustrates a flowchart for describing the aforementioned washing steps according to the present embodiment.

In step S51 of discharging matching liquid, the valve 72a of the liquid recovery line 72 is opened and the matching liquid 8 inside the compression device 1 is discharged.

In this case, a valve 71e and a valve 73a are closed beforehand.

Next, in step S52 of supplying a washing liquid, the valve 72a of the liquid recovery line 72 is closed, the valve 73a of the washing liquid supply line 73 is opened, and a washing liquid such as alcohol or hot water is supplied from the washing liquid supply line.

When the washing liquid has been filled into the compression device 1, the valve 73a is closed. At this time, the valve 71e is kept closed.

In this state, the washing liquid has replaced the matching liquid 8 as the liquid inside the compression device 1.

In this connection, when the flow rate at which the washing liquid is supplied is greater than the discharge liquid flow rate with respect to the liquid recovery, the valve 72a of the liquid recovery line 72 need not necessarily be closed.

Next, in step S53 of discharging the washing liquid, after a predetermined time has elapsed the valve 72a of the liquid recovery line 72 is opened, and the washing liquid after washing is discharged.

At this time, the valve 71e and the valve 73a are kept closed.

Alternatively, prior thereto, the valve 73a of the washing liquid supply line 73 and the valve 72a of the liquid recovery line 72 may be opened simultaneously, and the washing liquid may be allowed to flow continually.

Next, in step S54 of feeding matching liquid, the valve 72a of the liquid recovery line 72 is closed, the valve 71e of the matching liquid supply line 71 is opened, and the matching liquid 8 is fed.

Further, as necessary, in order to rinse out the washing liquid from inside the compression device 1, after step S54, step S53 may be performed, and thereafter step S54 may be performed again. It is thereby possible to inhibit the occurrence of residual washing liquid inside the compression device 1.

According to the above method, since it is possible to sterilize the inside of the compression device 1 and eliminate bacteria therefrom, the compression device 1 is made more hygienic.

Next, a method that actually compresses a subject with the compression device 1 is performed in the order of the following compressing steps (S61 to S64).

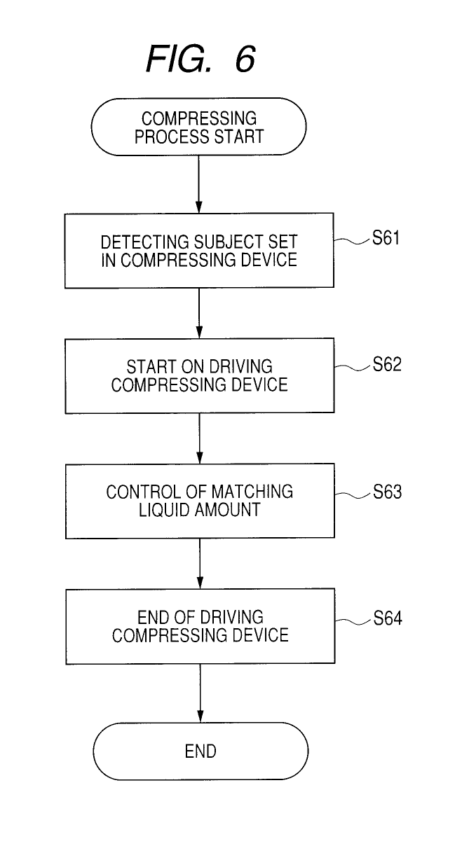

FIG. 6 illustrates a flowchart for describing the compressing process according to the present embodiment.

First, in step S61 which detects setting of a subject, a subject is set in the compression device in a state in which the matching liquid 8 has been filled inside the compression device 1.

At this time, the valve 72a of the liquid recovery line 72 is opened so that the matching liquid 8 does not overflow from the compression device 1 and the flow rate of the matching liquid 8 is controlled to adjust the liquid volume. Alternatively, the amount of the matching liquid 8 to be filled into the compression device 1 may be reduced in advance by an amount equal to the volume of the subject to be set therein.

Next, in step S62 of starting a compressing operation, the subject is compressed by the compression mechanism 5.

Next, in step S63 of controlling the liquid volume, the liquid volume (liquid surface) of the matching liquid 8 inside the compression device 1 is controlled.

Subsequently, in step S64 of ending the compressing operation, driving by the compression mechanism ends when a distance between the two compression plates 2 becomes a predetermined value. Alternatively, a sensor that measures a compressing force is provided, and the compression mechanism ends the driving when an output of the sensor becomes a predetermined value.

According to the above method, a subject can be compressed without causing the matching liquid 8 to overflow. After the compressing process is completed, photoacoustic measurement is performed.

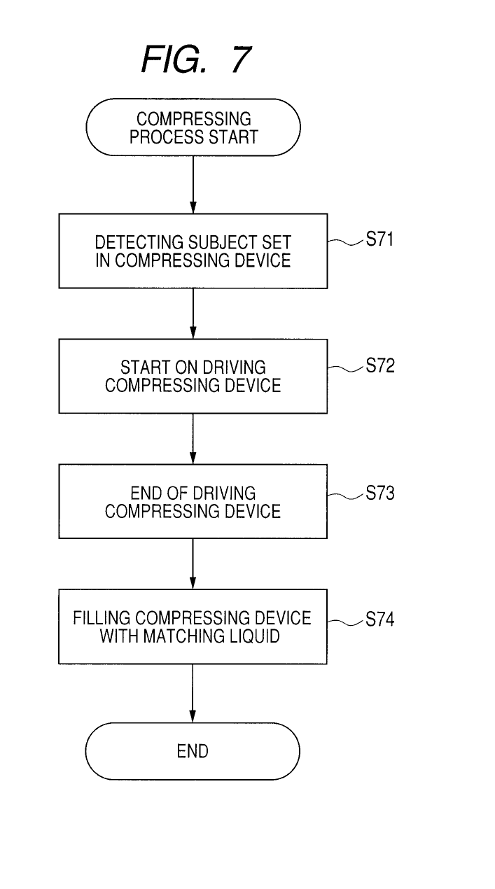

The above described compressing process is not limited to the method according to step S61 to step S64 that is described using FIG. 6, and may also be carried by a method that performs step S71 to step S74 in order as illustrated in FIG. 7.

In the case of the method illustrated in FIG. 7, first, in step S71 of detecting that a subject is set, matching liquid 8 inside the compression device 1 is emptied or is made a sufficiently small liquid volume in advance, and the subject is set in the compression device.

Next, in step S72 of starting a compressing operation, the subject is compressed by the compression mechanism 5.

Next, in step S73 of ending the compressing operation, driving by the compression mechanism ends when a distance between the two compression plates 2 becomes a predetermined value. Alternatively, a sensor that measures a compressing force is provided, and the compression mechanism ends the driving when an output of the sensor becomes a predetermined value.

Subsequently, in step S74 of filling the matching liquid, the matching liquid 8 is filled into the compression device 1.

Thus, in the case of performing step S71 to step S74, since the matching liquid 8 is not present inside the compression device 1 at a time of compressing (step S72), even if the flow rate for discharging the matching liquid 8 of the liquid recovery line 72 is insufficient, the matching liquid 8 does not overflow from the compression device 1.

### Embodiment 3

In Embodiment 3, a configuration example of a photoacoustic measurement apparatus on which the compression device of Embodiment 1 is mounted is described.

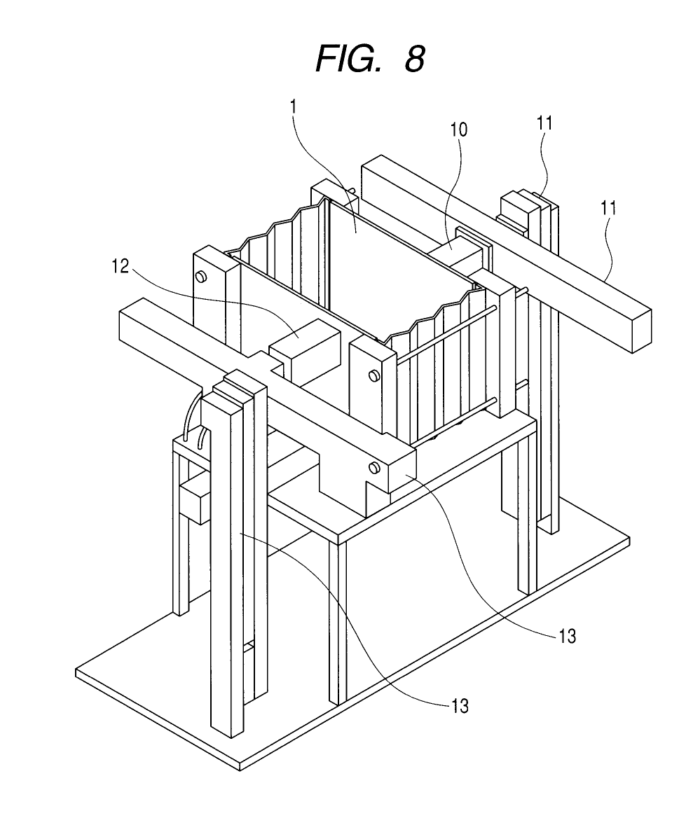

FIG. 8 illustrates the configuration of a photoacoustic measurement apparatus (mammography apparatus) (photoacoustic mammography, hereunder referred to as “PAM”).

The term “photoacoustic ” refers to acoustic waves that are generated when near infrared rays radiated to inside a subject are absorbed by tissues inside the subject and undergo local thermal expansion.

By receiving the photoacoustic waves with an ultrasound probe and extracting a signal unique to the photoacoustic waves, the tissue of the subject that absorbed the near infrared rays can be specifically imaged.

By radiating a wavelength that is easily absorbed by blood (hemoglobin) in particular among the subject tissues, an image of blood or a blood vessel can be specifically acquired, and in particular cancer angiogenesis can be imaged. As a result, the degree of progression of a cancer can be ascertained in more detail.

FIG. 8 illustrates an apparatus in which the principles of this type of photoacoustic measurement apparatus that radiates near infrared rays to inside a subject to cause the rays to be incident thereon receives photoacoustic signals generated inside the subject with an ultrasound probe, and displays a tissue image of inside the subject are applied to breast cancer screening.

In FIG. 8, the compression device described using FIG. 1 to FIG. 7 is denoted by reference numeral 1.

An illumination light optical system 10 is a system for irradiating laser beams of a wavelength from 650 nm to 1100 nm for generating photoacoustic waves from a subject (breast).

The illumination light optical system can be composed by an illumination light optical system for irradiating near infrared rays at a subject at least through at least one flat plate (through a compression plate) of the parallel flat plates.

In this connection, a laser light source and a route of illumination light from the laser light source to the illumination light optical system 10 are not shown in the drawing.

An illumination light scan unit 11 scans near infrared rays from the illumination light optical system and adjusts an irradiation position.

A probe 12 receives photoacoustic waves emitted from a subject (breast). A probe scan unit 13 is used for scanning the probe 12 and aligning the probe inside a predetermined region.

According to the photoacoustic measurement apparatus of the present embodiment, photoacoustic signals received by the probe 12 are amplified, subjected to A/D conversion, and undergo processing such as matching and adding, and envelope detection, and the resulting signals are used to perform three-dimensional image reconstruction.

The image information obtained by the three-dimensional image reconstruction processing is displayed on a monitor. The relevant processing portions and the monitor are not illustrated in FIG. 8.

Known methods may be applied as the methods for performing signal processing of photoacoustic signals to performing reconstruction of a three-dimensional image, and the present invention is not limited to a method described herein.

Matching liquid is filled inside the compression device 1 in order to match the acoustic impedances from the subject (breast) to the probe 12. Deaerated water can be used as the matching liquid, or the matching liquid may be an oil such as castor oil.

Next, the material of the compression plates 2 in the compression device 1 of the present embodiment will be described.

A resin such as polycarbonate or a glass such as quartz is suitable as the material of the compression plate 2 on the side of the illumination light optical system 10.

In order to match the acoustic impedances from the subject (breast) to the probe, the material of the other compression plate 2 on the side on which the probe is disposed can be a resin. In particular, polymethylpentene is suitable for forming this compression plate 2.

By mounting the compressing container 1 described above to a photoacoustic apparatus, time and labor required for cleaning can be reduced. Hence, the operating efficiency of the photoacoustic apparatus can be improved.

The compression device and the control method thereof of the present invention described in the foregoing can also be applied to an ultrasound apparatus, and not only a photoacoustic apparatus.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2009-010629, filed Jan. 21, 2009, which is hereby incorporated by reference herein in its entirety.

1. A compression device for compressing a subject that is used in ultrasonic measurement that receives ultrasonic waves from a subject to acquire biological information of the subject, comprising:
two compression plates that are used when compressing the subject, and that face each other in a vertical direction with respect to a horizontal plane;
a flexible member arranged so as to cover and seal an area between both side surfaces of and a bottom surface between the two compression plates, and so as not cover an area between top surfaces of the two compression plates such that an upper portion is open;
a compression mechanism that relatively changes a distance between the two compression plates to sandwich and compress the subject; and
a piping unit that supplies a matching liquid to a space portion surrounded by the two compression plates and the flexible member, and discharges the matching liquid that has been supplied.

2. The compression device according to claim 1, comprising a detection portion that detects a supply state of the matching liquid that is supplied to the space portion.

3. The compression device according to claim 2, comprising a control portion that controls supply of the matching liquid so that the matching liquid does not overflow from the space portion, based on an output of the detection portion.

4. The compression device according to claim 1, wherein the flexible member is any member of the group comprising an accordion-like member, a bellows, a resin film, or a rubber sheet.

5. The compression device according to claim 1, wherein the piping unit comprises at least one member of the group comprising:
a valve for controlling supply or discharge of the matching liquid;
a tank for storing the matching liquid;
a pump for feeding the matching liquid;
a pressure and flow rate adjustment valve for adjusting a pressure or a flow rate of the matching liquid;
a heater for heating the matching liquid;
a filter for removing impurities from the matching liquid; and
a de-aerated water generation apparatus that de-aerates dissolved gas included in the matching liquid.

6. The compression device according to claim 1, wherein the piping unit comprises a valve for controlling supply of a washing liquid.

7. A photoacoustic measurement apparatus that comprises a compression device which sandwiches and compresses a subject between two compression plates, and that compresses a subject using the compression device and detects as a photoacoustic signal an ultrasonic wave generated by a light irradiated at the subject,
wherein the compression device is a compressing unit comprising the compression device according to claim 1, and
the photoacoustic measurement apparatus further comprising:
an illumination light optical system for irradiating the light at a subject through the compression plate;
a probe that receives a photoacoustic signal generated from the subject through the compression plate; and
a processing portion that processes a photoacoustic signal received by the probe to reconstruct an image.

8. The photoacoustic measurement apparatus according to claim 7, wherein, of the two compression plates, a compression plate on a side on which the probe is disposed is formed by resin.

9. The photoacoustic measurement apparatus according to claim 8, wherein the resin comprises polymethylpentene.

10. A compressing control method of a compression device for compressing a subject that is used in ultrasonic measurement that receives ultrasonic waves from the subject to acquire biological information of the subject, comprising the steps of:
a washing process of washing inside of the space portion of the compression device according to of claims 1; and
after the washing process, a compressing process of sandwiching and compressing the subject with the two compression plates.

11. The compressing control method according to claim 10, the washing process comprising the steps of:
discharging a matching liquid that has been supplied to the space portion;
supplying a washing liquid to inside the space portion;
after supplying the washing liquid, discharging the washing liquid that has been supplied to inside the space portion; and
after discharging the washing liquid, supplying a matching liquid to inside the space portion.

12. The compressing control method according to claim 10, the compressing process comprising the steps of:
detecting setting of the subject inside the space portion;
starting a compressing operation by a compression mechanism in order to compress the subject;
controlling a liquid volume of a matching liquid that is filled to inside the space portion; and
ending the compressing operation by the compression mechanism.

13. The compressing control method according to claim 12, wherein:
based on an output of a detection portion that detects a supply state of the matching liquid that is supplied to the space portion,
the control of a liquid volume of the matching liquid controls a supply of the matching liquid so that the matching liquid does not overflow from the space portion.
